(19) United States
(12) Patent Application Publication
(10) Pub. No.: US 2021/0166804 A1
(43) Pub. Date: Jun. 3, 2021

(54) ANXIETY DETECTION USING WEARABLES

(71) Applicant: International Business Machines Corporation, Armonk, NY (US)

(72) Inventors: Emily Megan Metruck, Poughkeepsie, NY (US); Eli Michael Dow, Wappingers Falls, NY (US); Alejandro Demian Velasco, Austin, TX (US); Richard William Taggart, Poughkeepsie, NY (US)

(21) Appl. No.: 16/700,298

(22) Filed: Dec. 2, 2019

(51) Int. Cl. G16H 20/70; G16H 40/60

(52) U.S. Cl. CPC G16H 20/70; G16H 40/60

(57) ABSTRACT

Provided is a method, computer program product, and system for determining a user is experiencing anxiety when called upon to perform a task. A processor may monitor health data measurements received from a wearable device worn by a first user. In response to a prompt to complete a task from a second user, the processor may detect that the first user is experiencing anxiety based on analysis of the received health data measurements. The processor may determine the first user is experiencing difficulty completing the task as a result of the anxiety. The processor may classify the anxiety as a first anxiety type chosen from a plurality of anxiety types. In response to classifying the anxiety as the first anxiety type, the processor may output a recommendation to the second user for reducing the anxiety of the first user in order to assist the first user in completing the task.

### BACKGROUND

The present disclosure relates generally to the field of wearable electronic technology, and more specifically to determining that a user is experiencing anxiety when called upon to perform a task by another user.

In learning environments some students experience anxiety and/or stress when being called upon to perform a task in front of other students. In many instances, the student experiencing anxiety may not be able to successfully perform the task as a result.

### SUMMARY

Embodiments of the present disclosure include a method, computer program product, and system for determining that a user is experiencing anxiety when called upon to perform a task by another user. A processor may monitor health data measurements received from a wearable device worn by a first user. In response to a prompt to complete a task from a second user, the processor may detect that the first user is experiencing anxiety based on analysis of the received health data measurements. The processor may determine that the first user is experiencing difficulty completing the task as a result of the anxiety. The processor may classify the anxiety as a first anxiety type chosen from a plurality of anxiety types. In response to classifying the anxiety as the first anxiety type, the processor may output a recommendation to the second user for reducing the anxiety of the first user in order to assist the first user in completing the task.

The above summary is not intended to describe each illustrated embodiment or every implementation of the present disclosure.

### BRIEF DESCRIPTION OF THE DRAWINGS

The drawings included in the present disclosure are incorporated into, and form part of, the specification. They illustrate embodiments of the present disclosure and, along with the description, serve to explain the principles of the disclosure. The drawings are only illustrative of typical embodiments and do not limit the disclosure.

FIG. 1 illustrates a block diagram of a teaching assistant system, in accordance with embodiments of the present disclosure.

FIG. 2 illustrates a flow diagram of an example process for determining anxiety experienced by a user, in accordance with embodiments of the present disclosure.

FIG. 3 illustrates a high-level block diagram of an example computer system that may be used in implementing one or more of the methods, tools, and modules, and any related functions, described herein, in accordance with embodiments of the present disclosure.

FIG. 4 depicts a cloud computing environment according to an embodiment of the present invention.

FIG. 5 depicts abstraction model layers according to an embodiment of the present invention.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the disclosure.

### DETAILED DESCRIPTION

Aspects of the present disclosure relate to the field of wearable technology, and more particularly to determining that a user is experiencing anxiety when called upon to perform a task by another user. While the present disclosure is not necessarily limited to such applications, various aspects of the disclosure may be appreciated through a discussion of various examples using this context.

In learning environments some students experience anxiety and/or stress when being called upon to perform a task in front of other students (e.g., reading out loud, answering a question out loud, writing an answer on a chalkboard, etc.). In many instances, the student experiencing anxiety may not be able to successfully perform the task as a result. For example, a student that knows an answer to a question may still get the answer wrong when called upon by the teacher because of nervousness and/or anxiety.

In order to aid the student in completing the task successfully, a teacher may offer various forms of assistance and/or words of encouragement to help the student overcome their anxiety. Helping the student overcome their anxiety may improve the classroom experience for both the student and the teacher, thus resulting in a higher adoption rate of the classroom material. However, in some instances, the teacher may not be able to recognize students suffering from various forms of anxiety and/or how to provide appropriate assistance to the student for overcoming the specific anxiety type. As a result, some students may experience difficulty learning various subject matter within the classroom environment.

Embodiments of the present disclosure relate to determining if a user of wearable electronic technology is experiencing anxiety when called upon to perform a task by another user. In embodiments, a teaching assistant system may monitor health data measurements (e.g., oxygen level, pulse rate, blood pressure, galvanic sweat response (GSR), etc.) received from a wearable device (e.g., smart band, smart watch, smart sensor, etc.) worn by a first user to determine various physiological responses (e.g., nervousness, anxiety, stress, etc.) when being prompted to perform a task by a second user. For example, a teacher may call upon a student to read a passage from a textbook during a classroom exercise. The system may detect a change in the health data measurements (e.g., increase in pulse rate) and determine the user is experiencing anxiety (e.g., by comparing the health data measurements to an anxiety threshold) when attempting to complete the given task. For example, the student may be shown to be experiencing anxiety when their heart rate is determined to be greater than, e.g., 130 beats/min.

Once anxiety is detected, the system will determine if the first user has completed the given task successfully. If the system determines the first user is unsuccessful in completing the task, the system will determine the first user is experiencing difficulty resulting from the detected anxiety. Returning to the previous example, the system may determine (e.g., using natural language understanding (NLU), natural language processing (NLP), etc.) that the student is failing to read the passage from the textbook accurately (e.g., detection of stuttering, stammering, lengthy pauses between words/sentences, etc.) while anxiety is detected.

Once the first user is determined to be experiencing difficulty completing the given task, the system will classify what anxiety type the first user is experiencing. In embodiments, the system may utilize machine learning to determine a plurality of anxiety type classifications. For example, the system may analyze historical health data measurements and correlate the measurements with historical success rates for completing similar tasks. The system may compare various data trends and/or physiological responses to generate the plurality of anxiety type classifications. In embodiments, the system may utilize machine learning to determine various classifications and/or subcategories (e.g., anxiety resulting from different subject matters, types of tasks, etc.) of anxiety based on the historical data for each respective user. For example, anxiety types may be subdivided into categories based on anxiety experienced by the user with one or more subject matter (e.g., math, science, history, etc.).

For example, the system may differentiate between students that consistently experience public performance anxiety when called upon to answer a question during class as opposed to specific anxiety resulting from being unprepared to answer questions (e.g., data indicating the student only experiences anxiety when the student has not studied the subject matter). By utilizing machine learning, the system may determine over time what anxiety type each respective user is experiencing when being prompted to perform a given task. In this way, the system will be able to identify what the underlying cause is for creating the detected anxiety for the respective user and determine a way to reduce it.

Once the anxiety type is determined, the system may recommend to the second user a suggestion or technique on how to reduce the first user's anxiety in order to assist them in completing the given task successfully. Returning to the previous example, the system may recommend that the teacher suggest to the student to pause and take a few deep breathes between reading each sentence of the passage and/or offer words of encouragement to the student. In this way, the system may tailor recommendations to the determined anxiety type the student is experiencing in order to aid the student in overcoming their anxiety, allowing the student to successfully complete the task at hand.

In some embodiments, the user(s) must opt in to the system in order for the system to collect their information, and the user may determine which other users (e.g., which teacher(s)) can be provided recommendations based on the collected data. For example, during an initialization process, the system may inform the user of the types of data that it will collect (e.g., heart rate, blood pressure, etc.) and the reasons why the data is being collected. In these embodiments, the system will only start collecting the user information upon the user explicitly permitting the collection. Furthermore, the system may only collect the data that is necessary to provide anti-anxiety recommendations, the data may be anonymized and/or encrypted while in use, and the data may only be maintained as needed for providing the recommendations. If the user chooses to opt out of the system, any user information previously collected may be permanently deleted.

The aforementioned advantages are example advantages, and not all advantages are discussed. Furthermore, embodiments of the present disclosure can exist that contain all, some, or none of the aforementioned advantages while remaining within the spirit and scope of the present disclosure.

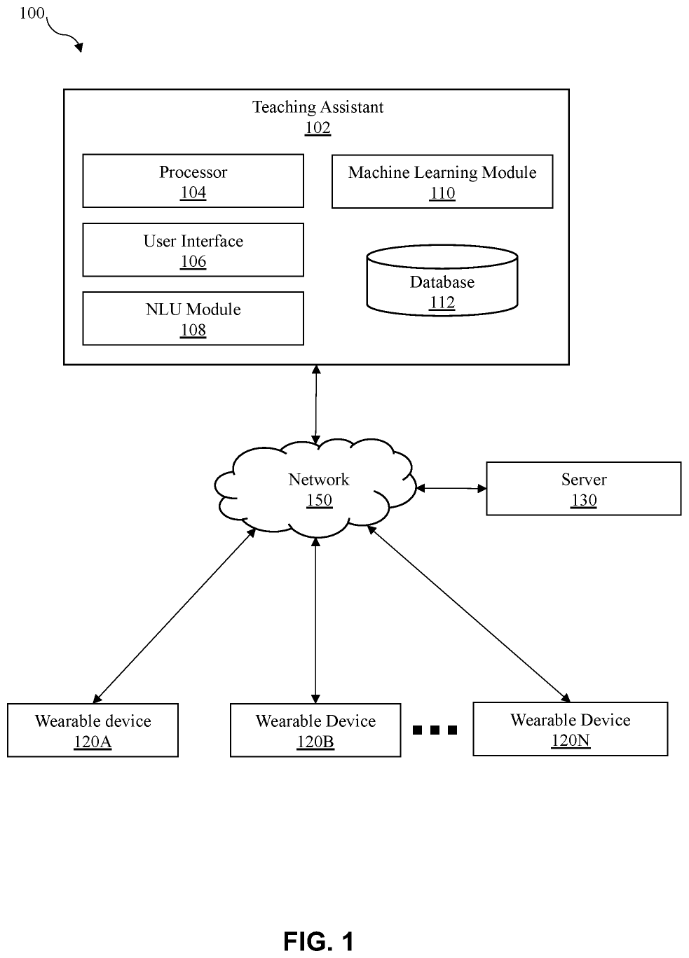

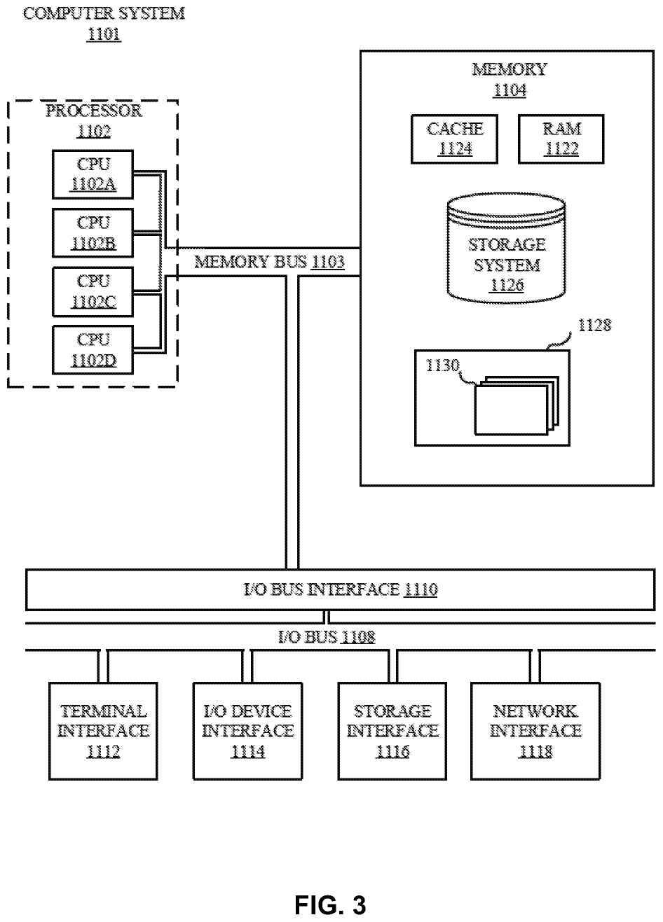

With reference now to FIG. 1, shown is a block diagram of teaching assistant system 100, in accordance with embodiments of the present disclosure. In the illustrated embodiment, teaching assistant system 100 includes teaching assistant 102 that is communicatively coupled to wearable device 120A, wearable device 120B, wearable device 120N (collectively referred to as wearable device(s) 120), and server 130 via network 150. Teaching assistant 102, wearable devices 120, and server 130 may be any type of computer system and may be substantially similar to computer system 1101 of FIG. 3. In embodiments, teaching assistant 102 may a standalone device or located on cloud technology. For example, teaching assistant 102 may be a virtual application accessed through a cloud computing network.

Wearable devices 120 may be any type of device (e.g., smart watch, smart band, smart sensor, etc.) configured to generate health data measurements associated with a user wearing the device. For example, wearable device 120A may be a smart sensor that generates health data measurements (e.g., heart rate data, galvanic skin response (GSR) data, oxygen level data, etc.) that are used to indicate the anxiety level of a first user wearing wearable device 120A, while wearable 120B may be a smart watch worn by a second user that generates health data measurements associated specifically to the second user. In embodiments, the health data measurements for wearable devices 120 may be sent to teaching assistant 102 to be analyzed for anxiety detection over network 150.

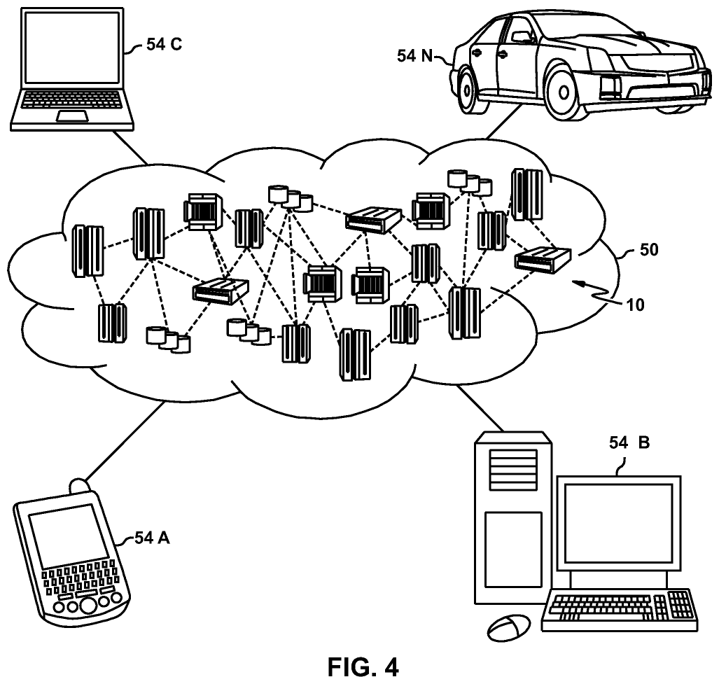

Network 150 may be any type of communication network, such as a wireless network or a cloud computing network. The network 150 may be substantially similar to, or the same as, cloud computing environment 50 described in FIG. 4. In some embodiments, the network 150 can be implemented using any number of any suitable communications media. For example, the network may be a wide area network (WAN), a local area network (LAN), a personal area network (PAN), an internet, or an intranet. In certain embodiments, the various systems may be local to each other, and communicate via any appropriate local communication medium.

For example, teaching assistant device 102 may communicate with wearable devices 120 and server 130 using a WAN, one or more hardwire connections (e.g., an Ethernet cable) and/or wireless communication networks. In some embodiments, the various systems may be communicatively coupled using a combination of one or more networks and/or one or more local connections. For example, teaching assistant device 102 may communicate with server 130 using a hardwired connection, while communication between wearable devices 120 and teaching assistant 102 may be through a wireless communication network.

In the illustrated embodiment, teaching assistant 102 includes processor 104, user interface 106, natural language understanding (NLU) module 108, machine learning module 110, and database 112.

In embodiments, user interface 106 is configured to display user profile information associated with each user and/or each wearable device 120. For example, user interface 106 may display a plurality of user profiles associated with a plurality of users participating in a learning session (e.g., students in a classroom environment, lecture, online classroom, etc.). Each user profile may display health data measurements received from a wearable device 120 worn by each respective user. For example, a teacher may monitor the health data measurements of all students participating in a class using user interface 106. In some embodiments, teaching assistant 102 may automatically identify users by recognizing wearable devices 120 (e.g., wearables communicatively coupling to the teaching assistant 102 via network 150) worn by the respective users when detected in the learning environment (e.g., physical classroom, virtual classroom, etc.). In some embodiments, teaching assistant system 100 may utilize various Internet of Things (IOT) devices (e.g., communicatively coupled cameras within the learning environment) to automatically identify users. For example, teaching assistant 102 may recognize various user (e.g., whether online users or users present in the classroom) via facial recognition using an IoT camera.

In embodiments, a new user profile may be generated for a new user automatically by teaching assistant 102 when recognizing a new wearable device 120 or through manual input by an administrator or teacher. User profiles may be stored on database 112 of teaching assistant 102 or server 130. Once users are identified by teaching assistant 102, health data measurements received from wearable devices 120 may be received and analyzed to determine if one or more users are experiencing anxiety when prompted to perform a task by another user.

In embodiments, NLU module 108 may monitor and/or analyze tasks given by the teacher for the students to perform in order to determine the appropriate response for successfully completing the given task. For example, the teacher may ask a student, “Who was the first president of the United States?” and provide an indication that the student is to respond out loud in front of the class. NLU module 108 may detect and analyze the given task to determine how the student can successfully complete the task at hand. For example, NLU module 108 may identify the subject matter of the question (e.g., presidential history of the United States) and determine the answer (e.g., George Washington) to the question by utilizing an information corpus (e.g., electronic books, current curriculum of the respective class, online resources, etc.) located on database 112. In some embodiments, database 112 may be a question and answer database that may be queried by teaching assistant 102 to determine the answers and/or responses for successfully completing tasks asked by the teacher.

Returning to the previous example, NLU module 108 may identify that the task includes an audible response from the student in order to be successfully completed. If the student is unable to complete the task by responding to the question audibly with the correct answer, NLU module 108 may determine the student has unsuccessfully completed the task. In some embodiments, the teacher may manually enter tasks (e.g., questions/answers for quizzes based on current curriculum, math problems, etc.) for the students to complete into database 112 of the teaching system 100. In embodiments, successful completion of the task may be determined by NLU module 108 or manually determined by the teacher. For example, a student having difficulty reading a passage in front of the class (e.g., detecting incorrectly read words, lengthy pauses, etc.) may be determined by NLU module 108 to be an unsuccessful attempt to complete the task. When attempts to complete the task are unsuccessful, teaching assistant 102 may determine the student is having difficulty completing the task due to the detected anxiety.

In embodiments, machine learning module 110 may monitor and/or analyze health data measurements received from wearable devices 120 to determine one or more anxiety types experienced by the user. Machine learning module 110 can utilize machine learning and/or deep learning, where algorithms or models can be generated by performing supervised, unsupervised, or semi-supervised training on historical health data measurements and historical success rates when performing various tasks in order to generate various classifications of anxiety types.

For example, the machine learning module 110 may utilize logical regression, decision tree classification, or support vector machine-based classification to determine when various tasks are completed successfully or unsuccessfully by the user, and whether the user was experiencing an indication of anxiety during performance of the given tasks. Over time, the machine learning module 110 can analyze the results to predict various types of anxieties experienced by the user when prompted to perform certain tasks. In embodiments, anxiety types may include subcategories based on anxiety experienced with various subject matter and/or genre of learning.

For example, a student may only experience public performance anxiety when asked to answer questions related to science because the student does not perform well in science class. Therefore, the anxiety type may be classified as public performance anxiety with a subcategory for science. Machine learning module 110 can utilize the anxiety type designation to make recommendations to the teacher on how to assist a student in reducing anxiety in order to complete a given task. For example, teaching assistant 102 may indicate to a science teacher that the respective student experiences anxiety in science class because they are not confident in their ability when answering questions due to poor understanding of the subject matter. Therefore, teaching assistant 102 may recommend to the teacher to give the student easier questions while further aiding and/or encouraging the student with coming up with the correct answers. In this way, teaching assistant 102 may help the teacher assess which students may need additional assistance when learning the respective material.

In another example, machine learning module 110 may determine that a student experiences performance anxiety only when performing math problems on a chalkboard because of both lack of confidence in understanding the subject matter and public performance anxiety. This may result in the student being unable to answer the problem successfully even when knowing the correct answer. Machine learning module 110 may classify this anxiety type as, e.g., math performance anxiety, and associate various recommendations on how to help the student overcome the specific anxiety type. For example, teaching assistant 102 may recommend to the teacher to allow the student to have more time to complete a math problem based on the student's math related anxiety and prior history with performing similar tasks. If solving the math problem requires breaking the problem into multiple parts, the teaching assistant 102 may also suggest that the teacher break the math problem into its constituent parts, each of which may be easier to answer, and have the student answer each part separately.

Machine learning module 110 can ingest the historical health data measurements and success rate/performance rate for completing tasks to generate insights, algorithms, and/or predictions related to making recommendations for reducing anxiety in each user. In embodiments, machine learning module 110 may correlate various recommendations for reducing anxiety to the respective anxiety types. In embodiments, the recommendations may be retrieved from a corpus of various recommendation and/or techniques for reducing anxiety stored on database 112.

In embodiments, machine learning module 110 may track which recommendations and/or techniques have been successful in helping the student reduce anxiety by analyzing the student's health data measurements after the recommendation has been given. Over time, with assistance and/or proper encouragement from the teacher (e.g., using the recommendations from the teaching assistant 102), the student may begin to experience less anxiety when called upon to perform tasks. Based on the changes in performance data, the machine learning module 110 may increase thresholds for the respective student based on successful completion of tasks. In this way, over time, machine learning module 110 can become more accurate in properly recognizing anxiety experienced by each user and adjust recommendations for the user as their progress changes when dealing with various forms of anxiety.

Machine learning algorithms can include, but are not limited to, decision tree learning, association rule learning, artificial neural networks, deep learning, inductive logic programming, support vector machines, clustering, Bayesian networks, reinforcement learning, representation learning, similarity/metric training, sparse dictionary learning, genetic algorithms, rule-based learning, and/or other machine learning techniques.

For example, the machine learning algorithms can utilize one or more of the following example techniques: K-nearest neighbor (KNN), learning vector quantization (LVQ), self-organizing map (SOM), logistic regression, ordinary least squares regression (OLSR), linear regression, stepwise regression, multivariate adaptive regression spline (MARS), ridge regression, least absolute shrinkage and selection operator (LASSO), elastic net, least-angle regression (LARS), probabilistic classifier, naïve Bayes classifier, binary classifier, linear classifier, hierarchical classifier, canonical correlation analysis (CCA), factor analysis, independent component analysis (ICA), linear discriminant analysis (LDA), multidimensional scaling (MDS), non-negative metric factorization (NMF), partial least squares regression (PLSR), principal component analysis (PCA), principal component regression (PCR), Sammon mapping, t-distributed stochastic neighbor embedding (t-SNE), bootstrap aggregating, ensemble averaging, gradient boosted decision tree (GBDT), gradient boosting machine (GBM), inductive bias algorithms, Q-learning, state-action-reward-state-action (SARSA), temporal difference (TD) learning, apriori algorithms, equivalence class transformation (ECLAT) algorithms, Gaussian process regression, gene expression programming, group method of data handling (GMDH), inductive logic programming, instance-based learning, logistic model trees, information fuzzy networks (IFN), hidden Markov models, Gaussian naïve Bayes, multinomial naïve Bayes, averaged one-dependence estimators (AODE), Bayesian network (BN), classification and regression tree (CART), chi-squared automatic interaction detection (CHAID), expectation-maximization algorithm, feedforward neural networks, logic learning machine, self-organizing map, single-linkage clustering, fuzzy clustering, hierarchical clustering, Boltzmann machines, convolutional neural networks, recurrent neural networks, hierarchical temporal memory (HTM), and/or other machine learning techniques.

FIG. 1 is intended to depict the representative major components of teaching assistant system 100. In some embodiments, however, individual components may have greater or lesser complexity than as represented in FIG. 1, components other than or in addition to those shown in FIG. 1 may be present, and the number, type, and configuration of such components may vary. Likewise, one or more components shown with teaching assistant system 100 may not be present, and the arrangement of components may vary.

For example, while FIG. 1 illustrates an example teaching assistant system 100 having a teaching assistant 102, three wearable devices 120, and a single server 130, suitable network architectures for implementing embodiments of this disclosure may include any number of teaching assistants, wearable devices, and servers. The various models, modules, systems, and components illustrated in FIG. 1 may exist, if at all, across a plurality of teaching assistant devices, wearable devices, and servers.

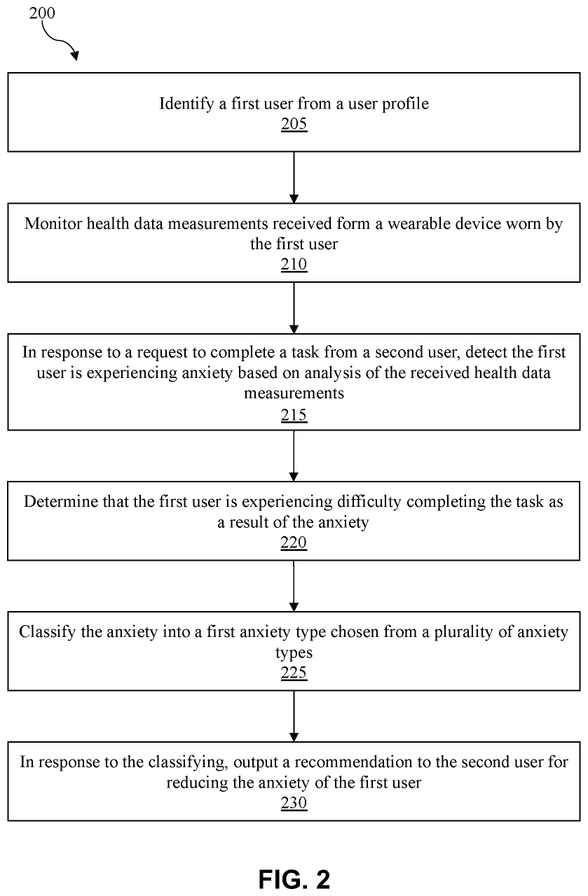

Referring now to FIG. 2, shown is a flow diagram of an example process 200 for determining anxiety experienced by a user, in accordance with embodiments of the present disclosure. The process 200 may be performed by processing logic that comprises hardware (e.g., circuitry, dedicated logic, programmable logic, microcode, etc.), software (e.g., instructions run on a processor), firmware, or a combination thereof. In some embodiments, the process 200 is a computer-implemented process. The process 200 may be performed by processor 104 exemplified in FIG. 1.

The process 200 begins by identifying a first user from a user profile. This is illustrated at step 205. The user profile may be utilized to identify each user participating in a learning session within an environment. For example, in a teaching environment, a teacher will determine which students are in the classroom by identifying each student by their user profile on user interface 106 of teaching assistant 102 detailed in FIG. 1.

The process 200 continues by monitoring health data measurements received from a wearable device worn by the first user. This is illustrated at step 210. The health data measurements may be any type of data used to indicate a user is experiencing anxiety. For example, the health data measurements may be a combination of heart rate data, oxygen level data, and/or GSR data received from one or more sensors disposed on the wearable device 120.

The process 200 continues by, in response to a prompt given to complete a task from a second user, identifying that the first user is experiencing anxiety based on analysis of the received health data measurements. This is illustrated at step 215. In embodiments, teaching assistant 102 may identify the user is experiencing anxiety by comparing the received health data measurements from the user's wearable device 120 to an anxiety threshold. The anxiety threshold may be based on historical health data measurements associated with the user when experiencing anxiety when performing various tasks.

In embodiments, teaching assistant 102 may correlate the anxiety threshold to each specific user so the system can accurately identify when the user is experiencing anxiety and/or stress. For example, historical health data measurements may show that the first user is experiencing anxiety when their heart rate falls within a certain range (e.g., 130-150 beats/min, >160 beasts/min, etc.), sweat is detected via a GSR sensor, and/or their oxygen levels increase or decrease. While for another user, the anxiety threshold may be higher or lower based off of their own individual historical health data measurements when performing tasks.

In some embodiments, the anxiety threshold may be a default threshold (e.g., for a new user) based on a plurality of other users (e.g., cohorts) having similar demographics (e.g., resting heart beats, blood pressure values, GSR data, heights, weights, anxiety indicators, etc.). In this way, teaching assistant 102 may identify anxiety experienced in new users placed within a cohort without the need for generating a specific anxiety threshold prediction for the new user. In embodiments, teaching assistant 102 may adjust the anxiety threshold for the new user as the system gathers health data measurements over a time period as the new user is asked to perform various tasks.

In embodiments, teaching assistant 102 may utilize NLU and/or NLP to determine what type of task was asked to be performed by the first user (e.g., answering a question, reading a passage out loud, writing on a chalkboard, etc.). For example, a teacher may ask a student, “What is a covalent bond?” during a chemistry class. The teaching assistant 102 may identify, using NLU module 108, what the task is and the proper response to complete it successfully. For example, to successfully complete the previous task example, the student must provide an audible answer indicating that a covalent bond is a chemical bond that involves the sharing of electron pairs between atoms.

The process 200 continues by determining that the first user is experiencing difficulty completing the task as a result of the anxiety. This is illustrated at step 220. In embodiments, teaching assistant 102 may utilize NLU to determine that the student has been unsuccessful in their attempt to complete the given task. For example, teaching assistant 102 may detect the student is pausing for a predetermined length of time (e.g., utilizing a time threshold generated from completion of similar tasks by other students) or has uttered various words (e.g., “I don't know” or “I'm unsure”) indicating the student is unsure of the answer to a question asked by the teacher.

In embodiments, teaching assistant 102 may compare the incorrect response/non-response given by the student to the correct response (e.g., obtained from querying database 112) and determine that the student is experiencing difficulty because they were unable to successfully completed the task. In some embodiments, the teacher may manually input into the system whether the first user is having difficulty completing the task (e.g., student was unable to complete a math problem on a chalkboard).

Once the first user is determined to be experiencing difficulty completing the task, the process 200 continues by classifying the determined anxiety into a first anxiety type chosen from a plurality of anxiety types. This is illustrated at step 225. For example, a student suffering from public performance anxiety may produce health data measurements indicating anxiety at all times when called to action to perform a task in front of the class regardless of whether they know the subject matter or not. Meanwhile, other users may not suffer from public performance anxiety and may only experience certain anxiety when they are unprepared for the class such that they cannot answer the question or complete a given task. Various classifications of anxiety types (e.g., public performance anxiety, unpreparedness/lack of knowledge of the subject matter, anxiety related to dyslexia when tasked to read out loud, and/or a combination of anxieties) may be determined by teaching assistant 102 by evaluating nuances between health data measurements and performance data utilizing machine learning (e.g., using machine learning module 110 detailed in FIG. 1).

For example, teaching assistant 102 may use machine learning to analyze historical health data measurements of the first user when experiencing anxiety when prompted to complete similar tasks. The system may correlate the historical health data measurements with historical success rates for completing similar tasks to generate a plurality of anxiety type thresholds associated with the plurality of anxiety types. Once the anxiety type thresholds are generated, the system may compare current health data measurements received from the wearable device to determine the specific classification of anxiety type being experienced by the first user. Teaching assistant 102 system may determine each user's physiological responses to being called upon to perform various tasks over time and determine anxiety types from performing data analysis. In this way, the system can distinguish between anxiety types being experienced by respective users.

In embodiments, the system may utilize various outputs (e.g., results from in class quiz, tests, assignments, etc.) generated by the student to determine if the detected anxiety is affecting the student's performance. For example, the system may determine by utilizing the student's grades if the student is just naturally anxious but otherwise performs well (e.g., student gets high marks for various tasks) or if the anxiety experienced by the student is affecting their performance (e.g., student is getting low marks for various tasks). In this way, the system may determine appropriate recommendations for reducing anxiety based on the correlation of the outputs and anxiety for each respective student. For example, a student that performs well in spite of their anxiety may only need some general anxiety therapy and/or avoidance of anxiety inducing situations and/or tasks. Alternatively, a student that performs poorly based off of the inputs and is shown to be suffering from anxiety may need both academic and anxiety remediation (e.g., extra tutoring, additional practice problems, anxiety therapy, etc.).

In response to classifying the determined anxiety into the first anxiety type, the process 200 continues by outputting a recommendation to the second user for reducing the anxiety of the first user. This is illustrated at step 230. For example, teaching assistant 102 may determine a student is suffering from public performance anxiety based on the student's current health data measurements matching historical responses of the student when asked to perform similar tasks. Teaching assistant 102 may recommend to the teacher to utilize various techniques for reducing anxiety in a person that suffers from similar public performance anxiety. For example, teaching assistant 102 may recommend to the teacher to remind the student to pause and take some deep breathes while completing a task (e.g. reading aloud, answering a question, etc.) in order to reduce the student's anxiety.

In embodiments, teaching assistant 102 may track and/or monitor what recommendations have worked for reducing anxiety of the user by monitoring the health data measurements after the recommendation has been attempted. In this way, over time the teacher may determine what techniques work best for each respective student based on historical response data. In some embodiments, health data measurements may indicate various techniques attempted may not adequately reduce the user's anxiety while in front of a class or during test taking. In such an instance, the system may recommend accommodating the student in another way (e.g., extended time for tests, private testing rooms, etc.).

In embodiments, if the first user has completed the task successfully, the process will return to step 210 and continue to monitor health data measurements from the wearable device 120 of the first user. In this way, the system will continually monitor and collect health data measurements in order to detect anxiety.

Referring now to FIG. 3, shown is a high-level block diagram of an example computer system 1101 that may be used in implementing one or more of the methods, tools, and modules, and any related functions, described herein (e.g., using one or more processor circuits or computer processors of the computer), in accordance with embodiments of the present disclosure. In some embodiments, the major components of the computer system 1101 may comprise one or more CPUs 1102, a memory subsystem 1104, a terminal interface 1112, a storage interface 1116, an I/O (Input/Output) device interface 1114, and a network interface 1118, all of which may be communicatively coupled, directly or indirectly, for inter-component communication via a memory bus 1103, an I/O bus 1108, and an I/O bus interface 1110.

The computer system 1101 may contain one or more general-purpose programmable central processing units (CPUs) 1102A, 1102B, 1102C, and 1102D, herein generically referred to as the CPU 1102. In some embodiments, the computer system 1101 may contain multiple processors typical of a relatively large system; however, in other embodiments the computer system 1101 may alternatively be a single CPU system. Each CPU 1102 may execute instructions stored in the memory subsystem 1104 and may include one or more levels of on-board cache. In some embodiments, a processor can include at least one or more of, a memory controller, and/or storage controller. In some embodiments, the CPU can execute the processes included herein (e.g., process 200).

System memory subsystem 1104 may include computer system readable media in the form of volatile memory, such as random access memory (RAM) 1122 or cache memory 1124. Computer system 1101 may further include other removable/non-removable, volatile/non-volatile computer system data storage media. By way of example only, storage system 1126 can be provided for reading from and writing to a non-removable, non-volatile magnetic media, such as a “hard drive.” Although not shown, a magnetic disk drive for reading from and writing to a removable, non-volatile magnetic disk (e.g., a “floppy disk”), or an optical disk drive for reading from or writing to a removable, non-volatile optical disc such as a CD-ROM, DVD-ROM or other optical media can be provided. In addition, memory subsystem 1104 can include flash memory, e.g., a flash memory stick drive or a flash drive. Memory devices can be connected to memory bus 1103 by one or more data media interfaces. The memory subsystem 1104 may include at least one program product having a set (e.g., at least one) of program modules that are configured to carry out the functions of various embodiments.

Although the memory bus 1103 is shown in FIG. 3 as a single bus structure providing a direct communication path among the CPUs 1102, the memory subsystem 1104, and the I/O bus interface 1110, the memory bus 1103 may, in some embodiments, include multiple different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface 1110 and the I/O bus 1108 are shown as single units, the computer system 1101 may, in some embodiments, contain multiple I/O bus interfaces 1110, multiple I/O buses 1108, or both. Further, while multiple I/O interface units are shown, which separate the I/O bus 1108 from various communications paths running to the various I/O devices, in other embodiments some or all of the I/O devices may be connected directly to one or more system I/O buses.

In some embodiments, the computer system 1101 may be a multi-user mainframe computer system, a single-user system, or a server computer or similar device that has little or no direct user interface, but receives requests from other computer systems (clients). Further, in some embodiments, the computer system 1101 may be implemented as a desktop computer, portable computer, laptop or notebook computer, tablet computer, pocket computer, telephone, smart phone, network switches or routers, or any other appropriate type of electronic device.

It is noted that FIG. 3 is intended to depict the representative major components of an exemplary computer system 1101. In some embodiments, however, individual components may have greater or lesser complexity than as represented in FIG. 3, components other than or in addition to those shown in FIG. 3 may be present, and the number, type, and configuration of such components may vary.

One or more programs/utilities 1128, each having at least one set of program modules 1130 may be stored in memory subsystem 1104. The programs/utilities 1128 may include a hypervisor (also referred to as a virtual machine monitor), one or more operating systems, one or more application programs, other program modules, and program data. Each of the operating systems, one or more application programs, other program modules, and program data or some combination thereof, may include an implementation of a networking environment. Programs/utilities 1128 and/or program modules 1130 generally perform the functions or methodologies of various embodiments.

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

Referring now to FIG. 4, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 10 and cloud computing environment 50 can communicate with any type of computerized device over any type of network and/or network addressable connection (e.g., using a web browser).

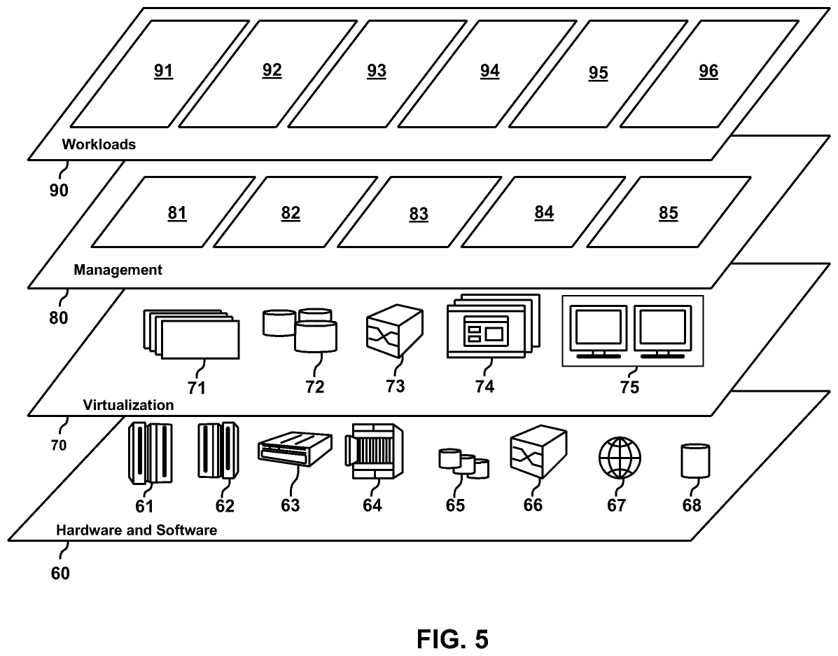

Referring now to FIG. 5, a set of functional abstraction layers provided by cloud computing environment 50 (FIG. 4) is shown. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and mobile desktops 96.

As discussed in more detail herein, it is contemplated that some or all of the operations of some of the embodiments of methods described herein may be performed in alternative orders or may not be performed at all; furthermore, multiple operations may occur at the same time or as an internal part of a larger process.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A computer-implemented method comprising:
monitoring health data measurements received from a wearable device worn by a first user;
in response to a prompt to complete a task from a second user, detecting that the first user is experiencing anxiety based on analysis of the received health data measurements;
determining that the first user is experiencing difficulty completing the task as a result of the anxiety;
classifying the anxiety as a first anxiety type chosen from a plurality of anxiety types; and
in response to classifying the anxiety as the first anxiety type, outputting a recommendation to the second user for reducing the anxiety of the first user in order to assist the first user in completing the task.

2. The computer-implemented method of claim 1, wherein detecting that the first user is experiencing anxiety comprises comparing the received health data measurements to an anxiety threshold.

3. The computer-implemented method of claim 2, wherein the anxiety threshold is based on historical health data measurements of the first user when experiencing anxiety.

4. The computer-implemented method of claim 1, wherein classifying the anxiety as the first anxiety type chosen from the plurality of anxiety types comprises:
analyzing, using machine learning, historical health data measurements of the first user when experiencing anxiety resulting from promptings to complete similar tasks;
correlating the historical health data measurements with historical success rates for completing the similar tasks to generate a plurality of anxiety type thresholds associated with the plurality of anxiety types; and
comparing current health data measurements to the plurality of anxiety type thresholds.

5. The computer-implemented method of claim 4, wherein the plurality of anxiety types are subdivided into categories based on anxiety experienced with one or more subject matter.

6. The computer-implemented method of claim 1, wherein the first anxiety type is classified as public performance anxiety.

7. The computer-implemented method of claim 1, wherein the first anxiety type comprises one or more subcategories of anxiety.

8. The computer-implemented method of claim 7, wherein the first anxiety type is classified as public performance anxiety and the one or more subcategories are classified as unpreparedness to successfully complete the task given by the second user.

9. The computer-implemented method of claim 1, wherein the first user is identified from an associated user profile.

10. The computer-implemented method of claim 1, wherein the recommendation is displayed on a user interface.

11. A system comprising:
a processor; and
a computer-readable storage medium communicatively coupled to the processor and storing program instructions which, when executed by the processor, cause the processor to perform a method comprising:
monitoring health data measurements received from a wearable device worn by a first user;
in response to a prompt to complete a task from a second user, detecting that the first user is experiencing anxiety based on analysis of the received health data measurements;
determining that the first user is experiencing difficulty completing the task as a result of the anxiety;
classifying the anxiety as a first anxiety type chosen from a plurality of anxiety types; and
in response to classifying the anxiety as the first anxiety type, outputting a recommendation to the second user for reducing the anxiety of the first user in order to assist the first user in completing the task.

12. The system of claim 11, wherein detecting that the first user is experiencing anxiety comprises comparing the received health data measurements to an anxiety threshold.

13. The system of claim 12, wherein the anxiety threshold is based on historical health data measurements of the first user when experiencing anxiety.

14. The system of claim 11, wherein classifying the anxiety as the first anxiety type chosen from the plurality of anxiety types comprises:
analyzing, using machine learning, historical health data measurements of the first user when experiencing anxiety when prompted to complete similar tasks;
correlating the historical health data measurements with historical success rates for completing the similar tasks to generate a plurality of anxiety type thresholds associated with the plurality of anxiety types; and
comparing current health data measurements to the plurality of anxiety type thresholds.

15. The system of claim 11, wherein the first anxiety type comprises one or more subcategories of anxiety.

16. A computer program product comprising a computer-readable storage medium having program instructions embodied therewith, wherein the computer-readable storage medium is not a transitory signal per se, the program instructions executable by a processor to cause the processor to perform a method comprising:
monitoring health data measurements received from a wearable device worn by a first user;
in response to a prompt to complete a task from a second user, detecting that the first user is experiencing anxiety based on analysis of the received health data measurements;
determining that the first user is experiencing difficulty completing the task as a result of the anxiety;
classifying the anxiety as a first anxiety type chosen from a plurality of anxiety types; and
in response to classifying the anxiety as the first anxiety type, outputting a recommendation to the second user for reducing the anxiety of the first user in order to assist the first user in completing the task.

17. The computer program product of claim 16, wherein detecting that the first user is experiencing anxiety comprises comparing the received health data measurements to an anxiety threshold.

18. The computer program product of claim 17, wherein the anxiety threshold is based on historical health data measurements of the first user when experiencing anxiety.

19. The computer program product of claim 16, wherein classifying the anxiety as the first anxiety type chosen from the plurality of anxiety types comprises:
analyzing, using machine learning, historical health data measurements of the first user when experiencing anxiety when prompted to complete similar tasks;
correlating the historical health data measurements with historical success rates for completing the similar tasks to generate a plurality of anxiety type thresholds associated with the plurality of anxiety types; and
comparing current health data measurements to the plurality of anxiety type thresholds.

20. The computer program product of claim 16, wherein the first anxiety type comprises one or more subcategories of anxiety.
